(19) United States
(12) Patent Application Publication
(10) Pub. No.: US 2012/0059407 A1
(43) Pub. Date: Mar. 8, 2012

(54) MULTI-PRONG FORCEPS

(76) Inventors: BRYCE Alan ISCH, Bluffton, IN (US); Nathan A. Winslow, Warsaw, IN (US)

(21) Appl. No.: 13/204,243

(22) Filed: Aug. 5, 2011

Related U.S. Application Data
(60) Provisional application No. 61/370,954, filed on Aug. 5, 2010.
(60) Provisional application No. 61/370,959, filed on Aug. 5, 2010.

(51) Int. Cl. A61B 17/28

(52) U.S. Cl. 606/205

(57) ABSTRACT

A surgical forceps comprising a first repositionable arm that includes a first prong extending distally therefrom and a second repositionable arm that includes a second prong extending distally therefrom, wherein each prong includes a distal flange that points inward and an interior surface of each prong is sloped. The surgical forceps may include a plurality of prongs for each arm. And the arms may be repositioned to reposition the prongs in order to bring the prongs closer together or farther apart. In exemplary form, the surgical forceps are adapted to grasp a semispherical device.

### CROSS REFERENCE TO RELATED APPLICATIONS

This application claims the benefit of U.S. Provisional Patent Application Ser. No. 61/370,954, filed Aug. 5, 2010, entitled “MULTI-PRONG FORCEPS,” and also claims the benefit of U.S. Provisional Patent Application Ser. No. 61/370,959, filed Aug. 5, 2010, entitled “MULTI-PRONG FORCEPS,” the disclosure of each is incorporated herein by reference.

### INTRODUCTION TO THE DISCLOSURE

The present disclosure is directed to surgical instruments and, specifically, to forceps. The exemplary forceps of the instant disclosure includes a novel prong or talon configuration that is particularly suited to grasp objects of varying sizes such as, without limitation, semispherical implant components for use with shoulder and hip arthroplasty procedures.

It is a first aspect of the present invention to provide a surgical forceps comprising: (a) a first repositionable arm that includes a first prong extending distally therefrom, the first prong including a base mounted to the first repositionable arm where the first prong extends laterally outward from the base and distally away from the base, the first prong including a distal flange that extends laterally inward toward the base but stops short of overlapping the base, the first prong including a concave interior surface and a convex exterior surface, wherein the flange includes a widthwise dimension that is substantially greater than a thickness of the flange in a vertical direction; (b) a second repositionable arm that includes a second prong extending distally therefrom, the second prong including a base mounted to the second repositionable arm where the second prong extends laterally outward from the base and distally away from the base, the second prong including a distal flange that extends laterally inward toward the base but stops short of overlapping the base, the second prong including a concave interior surface and a convex exterior surface, wherein the flange includes a widthwise dimension that is substantially greater than a thickness of the flange in a vertical direction; and, (c) a third prong extending distally from the second repositionable arm, the third prong including a base mounted to the second repositionable arm where the third prong extends laterally outward from the base and distally away from the base, the third prong including a distal flange that extends laterally inward toward the base but stops short of overlapping the base, the third prong including a concave interior surface and a convex exterior surface, wherein the flange includes a widthwise dimension that is substantially greater than a thickness of the flange in a vertical direction, where an end of the flange of the first prong is oriented generally in the direction of the end of the flange of the second and third prongs, where the convex exterior surface of the first prong is oriented generally away from the convex exterior surface of the second and third prongs, where the concave interior of the first prong is oriented generally toward the concave interior of the second and third prongs, and where an end of the flange of the third prong is oriented generally in the direction of the end of the flange of the second and third prongs.

In a more detailed embodiment of the first aspect, at least one of the first repositionable arm and the second repositionable arm includes a finger loop mounted thereto in order to reposition the arms with respect to one another. In yet another more detailed embodiment, the first repositionable arm and the second repositionable arm each includes a finger loop mounted thereto in order to reposition the arms with respect to one another. In a further detailed embodiment, the first repositionable arm is pivotally repositionable with respect to the second repositionable arm about a common axis, and the first repositionable arm and the second repositionable arm cooperate to form a box hinge. In still a further detailed embodiment, the second and third prongs are rotationally offset from one anther between ten and ninety degrees. In a more detailed embodiment, the first and second prongs are rotationally offset from one anther between forth-five and three hundred and twenty ninety degrees. In a more detailed embodiment, the forceps further includes a fourth prong extending distally from the first repositionable arm, the fourth prong including a base mounted to the first repositionable arm where the fourth prong extends laterally outward from the base and distally away from the base, the fourth prong including a distal flange that extends laterally inward toward the base but stops short of overlapping the base, the fourth prong including a concave interior surface and a convex exterior surface, wherein the flange includes a widthwise dimension that is substantially greater than a thickness of the flange in a vertical direction. In another more detailed embodiment, the first and fourth prongs are rotationally offset from one anther between ten and ninety degrees. In yet another more detailed embodiment, the concave interior surface of the first prong includes a planar segment acutely angled with respect to a longitudinal axis extending through the first arm and, the concave interior surface of the second prong includes a planar segment acutely angled with respect to a longitudinal axis extending through the second arm. In still another more detailed embodiment, the concave interior surface of the second prong includes a planar segment acutely angled with respect to a longitudinal axis extending through the first arm and, the concave interior surface of the third prong includes a planar segment acutely angled with respect to a longitudinal axis extending through the second arm.

In yet another more detailed embodiment of the first aspect, the concave interior surface of the first prong includes a planar segment acutely angled with respect to a longitudinal axis extending through the first arm and, the concave interior surface of the second prong includes a planar segment acutely angled with respect to a longitudinal axis extending through the second arm. In still another more detailed embodiment, the concave interior surface of the first prong includes a first planar segment adjoining a second planar segment adjoining a convex interior surface. In a further detailed embodiment, the concave interior surface of the second prong includes a first planar segment adjoining a second planar segment adjoining a convex interior surface and, the convex interior surfaces of the first and second prongs face one another.

It is a second aspect of the present invention to provide a forceps comprising: (a) a first arm and a second arm pivotally connected to each other, each of the first and second arms having opposing ends; (b) a first jaw coupled to the first arm, the first jaw including at least one laterally outward bowed prong at least partially defining an interior concavity, the at least one laterally outward bowed prong includes a distal flange extending medially in a first plane; and, (c) a second jaw coupled to the second arm opposite the second finger hold, the second jaw including at least one medially outward bowed prong at least partially defining the internal concavity, the at least one medially outward bowed prong includes a distal flange extending laterally in the first plane.

In a more detailed embodiment of the second aspect, the forceps further includes a first finger hold and a second finger hold, each of the first and second finger holds being coupled to one of the opposing ends of the first and second arms, where the first finger hold and the second finger hold are repositionable relative to one another to pivotally reposition the first arm with respect to the second arm. In yet another more detailed embodiment, the first arm and second arm are pivotally connected to each other using a box hinge. In a further detailed embodiment, the at least one laterally outward bowed prong of the first jaw includes a concave interior surface and this concave interior surface includes a convex segment and, the at least one laterally outward bowed prong of the second jaw includes a concave interior surface and this concave interior surface includes a convex segment.

It is a third aspect of the present invention to provide a forceps comprising: (a) a first arm mounted to and repositionable with respect to a second arm; (b) a first prong coupled to the first arm at a first junction having an X-axis that is perpendicular to a Y-axis that is perpendicular to a Z-axis that is itself perpendicular to the X-axis, the first prong extending proximally away from the first junction along the X-axis in a positive x-direction and laterally away from the first junction in the Y-axis in a positive y-direction, the first prong also including a distal flange that extends laterally toward the first junction in the Y-axis in a negative y-direction but does not overlap the first junction; and, (c) a second prong coupled to the second arm at a second junction having the same coordinate system as the first prong, the second prong extending proximally away from the second junction along the X-axis in the positive x-direction and laterally away from the junction in the Y-axis in the negative y-direction, the second prong also including a distal flange that extends laterally toward the second junction in the Y-axis in the positive y-direction but does not overlap the second junction, where the distal flange of the first prong extends toward the distal flange of the second prong.

In a more detailed embodiment of the third aspect, the forceps further includes a third prong coupled to the first arm at a third junction having the same coordinate system as the first prong, the third prong extending proximally away from the third junction along the X-axis in the positive x-direction and laterally away from the junction in the Y-axis in the positive y-direction, the third prong also including a distal flange that extends laterally toward the third junction in the Y-axis in the negative y-direction but does not overlap the third junction. In yet another more detailed embodiment, the forceps further includes a fourth prong coupled to the second arm at a fourth junction having the same coordinate system as the first prong, the fourth prong extending proximally away from the fourth junction along the X-axis in the positive x-direction and laterally away from the junction in the Y-axis in the negative y-direction, the fourth prong also including a distal flange that extends laterally toward the fourth junction in the Y-axis in the positive y-direction but does not overlap the fourth junction, wherein the distal flanges of the first and third prongs extend toward the distal flanges of the second and fourth prongs.

It is a fourth aspect of the present invention to provide a surgical forceps comprising a first leg repositionable with respect to a second leg, the first leg having mounted thereto a first hook, the first hook including a convex exterior portion and a concave interior portion, the concave interior portion of the first hook partially defining an internal cavity, the second leg having mounted thereto a second hook, the second hook including a convex exterior portion and a concave interior portion, the concave interior portion of the second hook partially defining the internal cavity, where the concave interior portions of the first and second hooks face one another.

In a more detailed embodiment of the fourth aspect, the concave interior portion of at least one of the first hook and the second hook includes a convex interior portion. In yet another more detailed embodiment, the first leg has mounted thereto a third hook, the third hook including a convex exterior portion and a concave interior portion, the concave interior portion of the third hook partially defining the internal cavity and, the first hook and third hook are radially offset from one another between ten and ninety degrees. In a further detailed embodiment, the concave interior portions of the first and third hooks face the convex interior portion of the second hook. In still a further detailed embodiment, the second leg has mounted thereto a fourth hook, the fourth hook including a convex exterior portion and a concave interior portion, the concave interior portion of the fourth hook partially defining the internal cavity and, the second hook and fourth hook are radially offset from one another between ten and ninety degrees. In a more detailed embodiment, the first hook and the fourth hook are radially offset from one another between eighty and two hundred and eighty degrees. In a more detailed embodiment, the concave interior portions of the first and third hooks face the convex interior portions of the second and fourth hooks.

### BRIEF DESCRIPTION OF THE DRAWINGS

FIG. 1 is an elevated perspective view of a first exemplary forceps in accordance with the instant disclosure.

FIG. 2 is a frontal view of an exemplary two prong jaw of FIG. 1.

FIG. 3 is an overhead view of the exemplary two prong jaw of FIG. 1.

FIG. 4 is a cross-sectional view taken along line 3-3 of FIG. 3.

FIG. 5 is a profile view of the exemplary single prong jaw of FIG. 1.

FIG. 6 is an elevated perspective view of the exemplary forceps of FIG. 1, from the proximal end.

FIG. 7 is an elevated perspective, profile view of the exemplary forceps of FIG. 1.

FIG. 8 is a magnified, elevated perspective view of the exemplary jaws of FIG. 1 grasping a semispherical prosthetic implant.

FIG. 9 is a proximal end view of the exemplary forceps of FIG. 1 grasping a semispherical prosthetic implant.

FIG. 10 is a profile, exploded view of an exemplary prosthetic shoulder assembly.

FIG. 11 is an elevated perspective view of a second exemplary forceps in accordance with the instant disclosure.

FIG. 12 is an elevated perspective view of the second exemplary forceps of FIG. 11 shown grasping a semispherical implant.

FIG. 13 is a profile view of the second exemplary forceps of FIG. 11 shown grasping a semispherical implant.

FIG. 14 is a magnified, profile view of the distal end of the second exemplary forceps of FIG. 13 shown grasping a semispherical implant.

FIG. 15 is a magnified, elevated perspective view of the distal end of the second exemplary forceps of FIG. 13 shown grasping a semispherical implant.

FIG. 16 is a bottom view of the semispherical implant being grasped by the exemplary forceps of FIG. 11.

### DETAILED DESCRIPTION

The exemplary embodiments of the present disclosure are described and illustrated below to encompass forceps surgical instruments, and related methods for using and fabricating forceps surgical instruments. Of course, it will be apparent to those of ordinary skill in the art that the embodiments discussed below are exemplary in nature and may be reconfigured without departing from the scope and spirit of the present disclosure. However, for clarity and precision, the exemplary embodiments as discussed below may include optional steps, methods, and features that one of ordinary skill should recognize as not being a requisite to fall within the scope of the present invention.

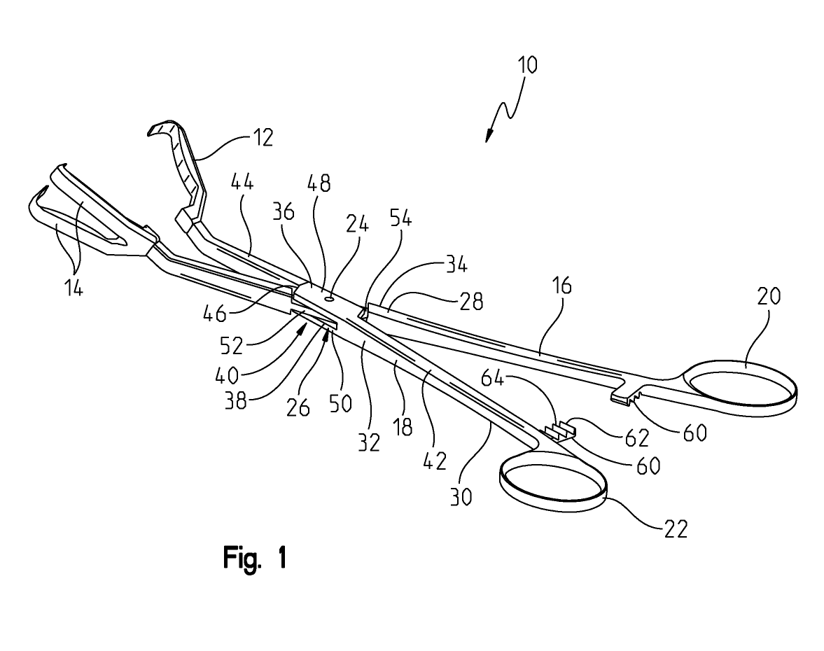

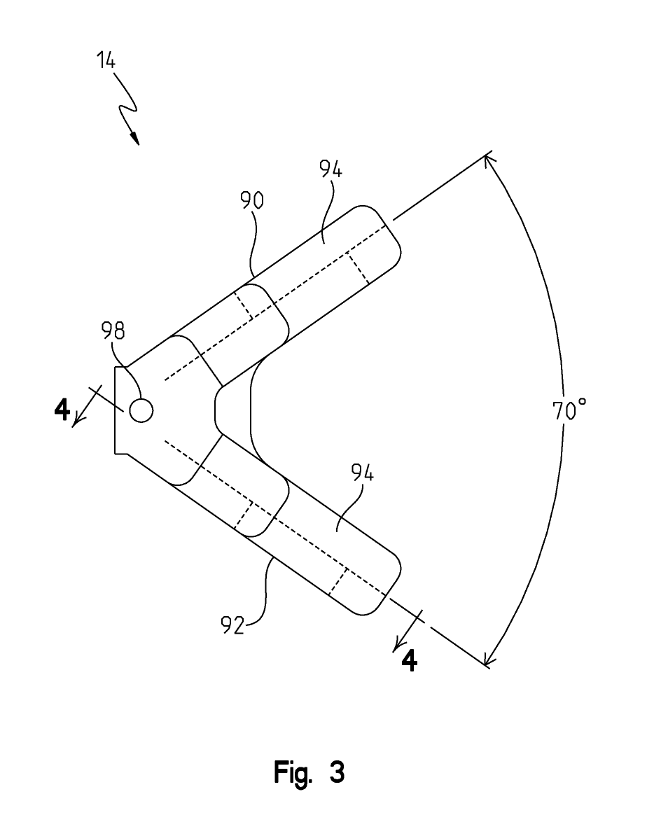

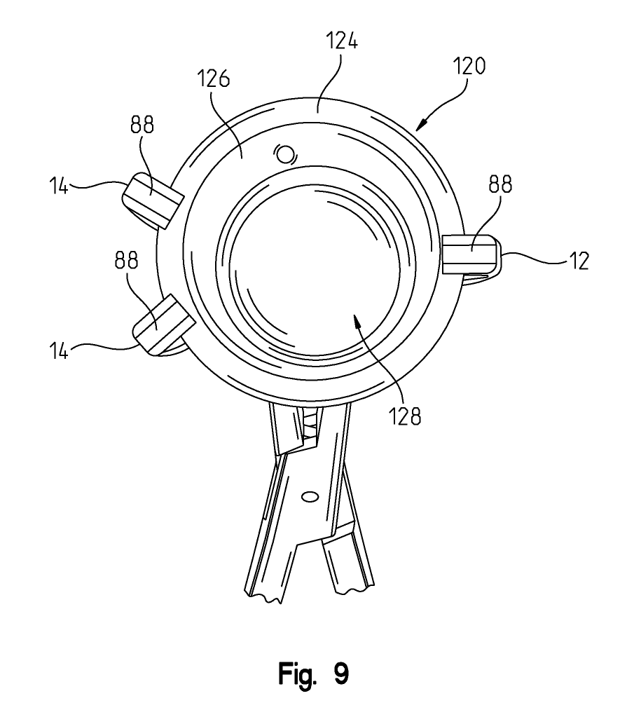

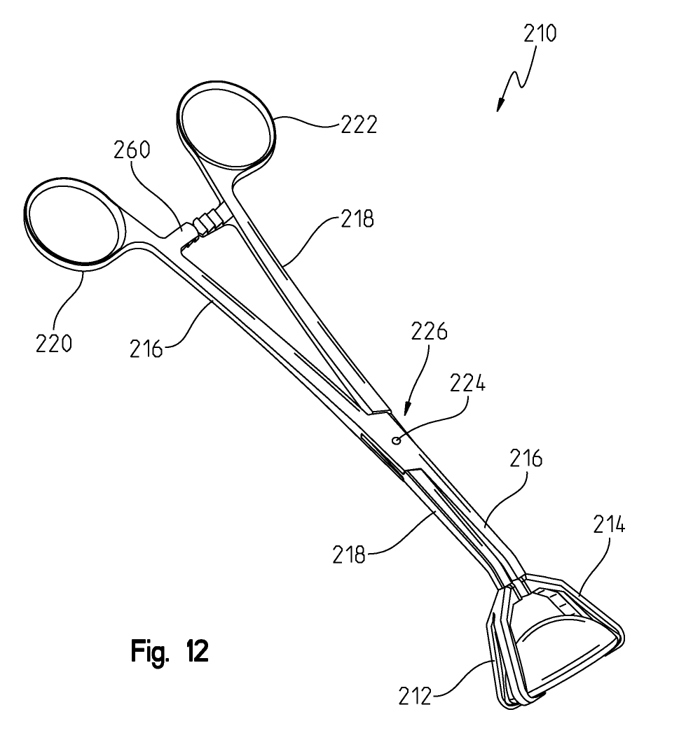

Referencing FIGS. 1-3 and 9-12, an exemplary surgical instrument 10, generally referred to hereafter as a forceps, includes opposed jaws 12, 14 that are repositionable with respect to one another to accommodate and retain different sized objects. Each jaw 12, 14 is mounted to a proximal end of a respective arm 16, 18 that terminates in finger holds 20, 22 at a proximal end. The arms 16, 18 are pivotally coupled to one another via a pivot pin 24 in order to allow the relative positions of the jaws 12, 14 to be changed by manipulating the finger holds 20, 22. In this exemplary embodiment, the arms 16, 18 are pivotally coupled to one another via a box hinge 26. However, those skilled in the art will understand that hinge arrangements other than a box hinge may be used with the exemplary forceps 10 such as, without limitation, a scissor hinge.

Each arm 16, 18 includes a rectangular cross-section extending between the finger holds 20, 22 and box hinge 26, as well as between the box hinge and the jaws 12, 14. The cross-section of the arms 16, 18 changes along the length of each arm. By way of example, the cross-section of each arm 16, 18 at the proximal end, proximate the finger holds 20, 22, is dominated by larger top and bottom surfaces 28, 30 in comparison to smaller left and right side surfaces 32, 34. But the cross-section changes so that proximate the box hinge 26, the top and bottom surfaces 28, 30 are only slightly larger than the left and right side surfaces 32, 34. On a proximal end of the box hinge 26, the top and bottom surfaces 28, 30 have approximately the dimensions as the left and right side surfaces 32, 34, while these dimensions decrease proportionally until reaching the distal end proximate the jaws 12, 14.

The box hinge 26 comprises a female portion that includes corresponding rails 36, 38 of the second arm 18 that define a gap 40 therebetween. Each rail 36, 38 is integrally mounted to proximal and distal segments 42, 44 of the second arm 18 and is operative to longitudinally offset the proximal segment from the distal segment. Both rails 36, 38 include an obtuse angled corner 46 that is laterally offset from the proximal and distal segments. Though not required, each rail 36, 38 includes substantially planar top and bottom surfaces 48, 50 that are separated from one another by a constant dimension, evidenced by a uniform material thickness between the top and bottom surfaces. The gap 40 between the rails 36, 38 is partially occupied by a male portion of the first arm 16 that includes a beam 52 formed by reducing the thickness of the first arm along a predetermined portion of the arm. This reduction in thickness in the first arm 16 may be accomplished by forging or grinding away material to form two generally rectangular cutouts 54. In this exemplary embodiment, the depth of the cutouts 54 is approximately the same as the thickness (i.e., distance between top and bottom surfaces 48, 50 of each rail) of each rail 36, 38. Both the rails 36, 38 and the beam 52 include a through hole (not shown) that receives the pivot pin 24, thereby allowing the rails to pivot with respect to the beam and, thus, the arms 16, 18 to pivot with respect to one another. As discussed previously, the proximal and distal segments 42, 44 of the second arm 18 are longitudinally offset, whereas the first arm 16 is generally longitudinally aligned. As a result, when the arms 16, 18 are pivoted with respect to one another to close the jaws 12, 14, the distal ends of the arms 16, 18 are substantially parallel and adjacent.

Opposite the proximal ends, both arms 16, 18 include serrated lugs 60 that protrude toward one another in an inverted orientation. The serrated lugs 60 include a series of ramps 62 interposed by detent surfaces 64 that provide for a ratcheting action when the lugs engage one another. When the serrated lugs 60 engage each other, the lugs are operative to maintain the jaws 12, 14 in a predetermined position. As will be discussed in more detail hereafter, the serrated lugs 60 are operative to lock the jaws 12, 14 in compression around various sized objects such as, without limitation, a semispherical implant. It should also be noted that the serrated lugs 60 may be replaced or supplemented by any locking mechanism useful to lock the jaws 12, 14 in place.

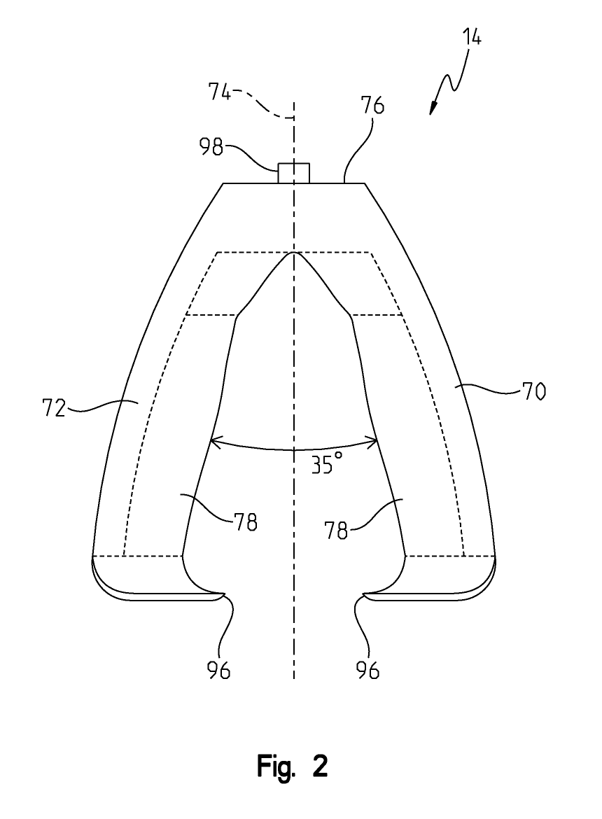

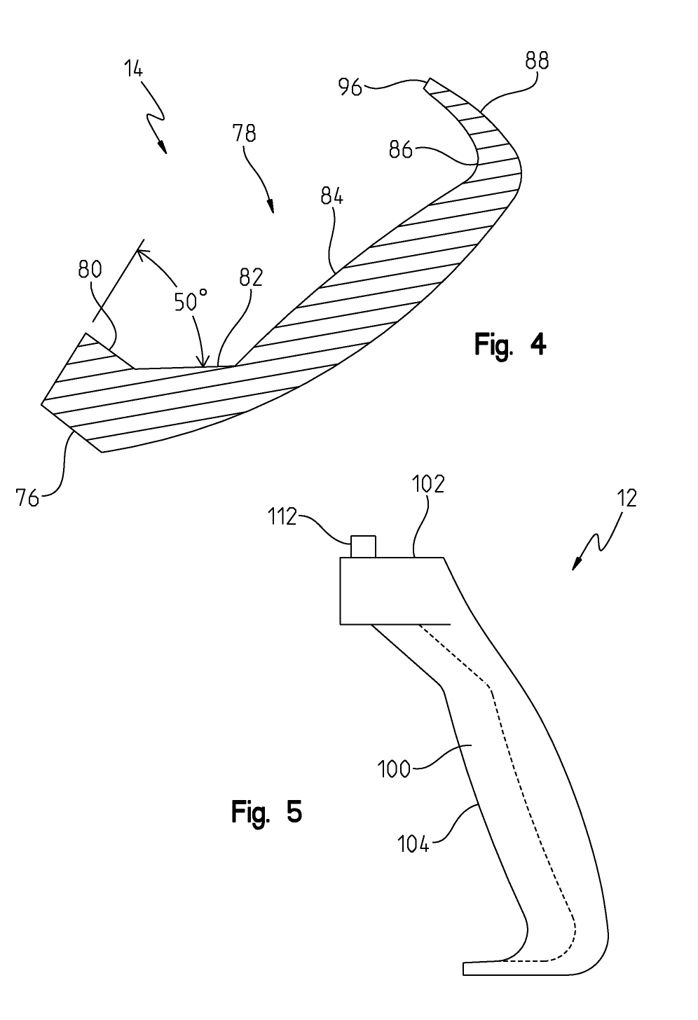

Referencing FIGS. 2-4, the second jaw 14 includes a pair of prongs 70, 72 that are radially offset approximately 70 degrees with respect to one another, and angled approximately 35 degrees with respect to a vertical axis 74 that extends through a substantially planar common base 76. A convex interior surface 78 of each prong 70, 72 includes a number of distinct sections. A first section 80 is generally planar and comprises a reference plane. A second section 82, which interposes the first section 80 and the third section 84, is generally planar and angled approximately 50 degrees with respect to the first section. The third section 84 is convex and arcuately shaped in order for the prongs 70, 72 to accommodate semispherical objects having different diameters. At the base of the third section 84, opposite the second section 82, is a more severe arcuate surface 86 that extends outward, away from the third section to create a flange 88. This flange 88 extends inward from the interior surface 78. In exemplary form, the flat interior surface of the flange is acutely angled with respect to the third section 84

Perpendicular side surfaces 90, 92 extend from the interior surface 78 and round over to join a convex, arcuate external surface 94, generally opposite the interior surface. The external surface 94 and interior surface 78 eventually converge at the tip 96 of the flange 88. In order to join the second jaw 14 to the first arm 16, the second jaw includes a projection 98 that extends from a base 76, opposite the flange 88 end of the prongs 70, 72, that is received within a corresponding opening (not shown) at the proximal end of the first arm 16. It should be noted that the projection 98 is substantially coaxial with the vertical axis 74 and that the common base 76 is parallel with the first section 80 of the interior surface 78.

Referring to FIG. 5, in contrast to the second jaw 14, the first jaw 12 includes a single prong 100 having a substantially planar base 102. An interior surface 104 of this prong 100 is generally the same as the interior surface 78 of the prongs 70, 72 discussed immediately above. Consequently, for purposes of brevity, a discussion of the interior surface 104 of this single prong 100 has been omitted. Likewise, the side and external surfaces of this single prong 100 are generally the same as the side and external surfaces 90, 92, 94 discussed above for the dual prongs 70, 72. Thus, for purposes of brevity, a discussion of the external surfaces of this single prong 100 has been omitted. And the first jaw 12 also includes a projection 112 that extends from the base 102, opposite the flange end of the prong, that is received within a corresponding opening (not shown) at the proximal end of the second arm 18. However, while the prongs 70, 72 of the second jaw 14 were radially offset approximately 15 degrees from the vertical axis 74, the prong 100 of the first jaw 12 is radially aligned with the vertical axis 74.

The forceps 10 may be manufactured from any material. In exemplary form, the forceps 10 are manufactured from a metal in order to create a reusable tool. Exemplary metals include, without limitation, stainless steel, titanium, and nickel. Alternately, the forceps 10 may be manufactured using a plastic material for immediate disposal after one use. And the forceps may be also manufactured from a ceramic.

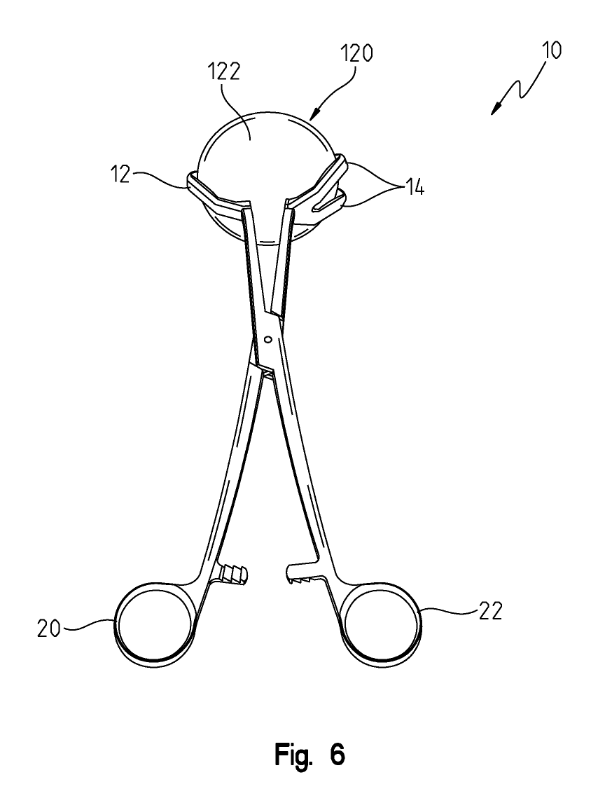

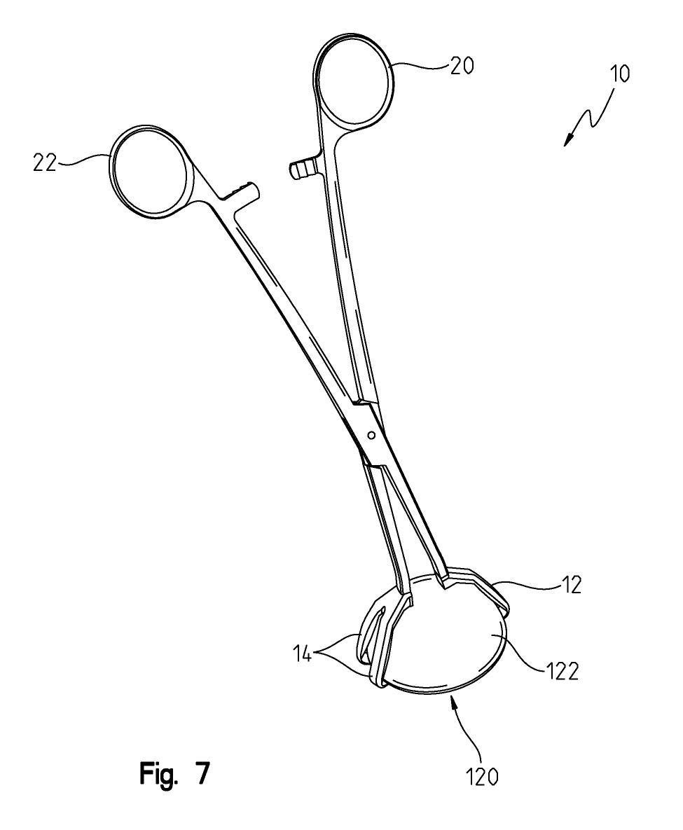

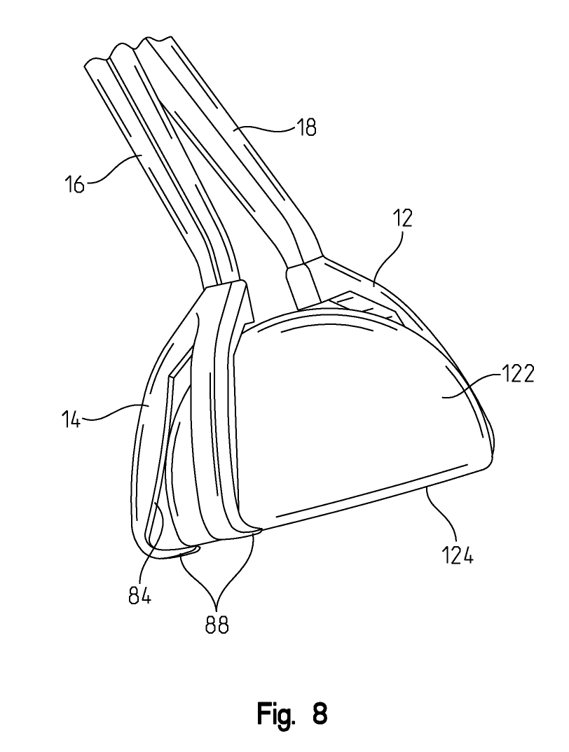

Referring to FIGS. 1, and 6-10, in use, the exemplary forceps 10 are opened so that the jaws 12, 14 are spaced apart from one another in order to grasp an object 120. In exemplary form, this object may comprise a semispherical or spherical implant head 120, such as those used for hip joint replacement and shoulder joint replacement surgeries. The finger holds 20, 22 are respectively adapted to accept a finger and thumb of a user (not shown) so that the finger holds 20, 22 are moved farther away from one another in order to correspondingly move the jaws 12, 14 away from one another. After the jaws 12, 14 are sufficiently spaced apart to accommodate the object 120 in question, the jaws are brought closer together to secure the object between the jaws.

In the context of a semispherical implant head 120, the spherical part 122 of the implant head is oriented to face the proximal end of the forceps 10 so that the spherical part contacts the third section 84 of each prong interior surface 78, 104. In exemplary form, the underside of the implant head is relatively flat with a rounded-over circumferential lip 124 being raised and outset from an inset, and depressed circumferential ring 126. This circumferential ring 126 generally defines an opening 128 that receives a disc 129 of a taper adapter 130 having a tapered projection 132 extending therefrom. The tapered projection 132 is received within a cavity 140 of a prosthetic stem 142 that includes a shaft 144 extending away from the cavity. In this manner, the implant head 120 is coupled to the taper adapter 130, which is coupled to the prosthetic stem 142. In exemplary form, the prosthetic stem 142, taper adapter 130, and implant head 120 are part of a shoulder implant assembly 150.

After the implant head 120 is positioned between the jaws 12, 14, so that the spherical part 122 initially contacts the third section 84 of each prong interior surface 78, 104, the jaws are moved toward one another to compress the implant head. When the spherical part 122 contacts the third sections 84 of the prongs 70, 72, 100, a tapered wedge is created that forces the circumferential lip 124 toward and eventually against the distal flanges 88, caused by compressing the jaws 12, 14 circumferentially around the implant head 120. Specifically, the circumferential lip 124 is seated upon the distal flanges 88, while the spherical part initially contacts the third section 84 of each prong interior surface 78, 104. In general, referencing the circumference of the implant head as a circle proximate the circumferential lip 124, the prongs are oriented and centered at approximately 0 degrees for the single prong 100 of the first jaw 12, and at approximately 165 and 195 degrees for the dual prongs 70, 72 of the second jaw 14.

Depending upon the size of the implant head 120, the jaws 12, 14 may retain the implant head in compression when the serrated lugs 60 have engaged one another. When the jaws 12, 14, and thus the finger holds 20, 22, are moved toward one another in close enough proximity, the sloped surfaces 62 of the serrated lugs 60 lugs ride against one another so that the detent surfaces 64 can engage one another and inhibit movement of the jaws 12, 14 (and finger holds 20, 22) further away from one another. At the same time, the angled surfaces 62 and detent surfaces 64 allow the jaws 12, 14 and the finger holds 20, 22 to be moved closer to one another (presuming no object between the jaws forecloses further movement) until reaching an endpoint. As a result, the serrated lugs 60 allow the jaws 12, 14 to be locked in a compression position, within a predetermined range of motion, while the implant head 120 is between the jaws 12, 14.

In order to release the implant head 120 from the jaws 12, 14, the finger holds 20, 22 are manipulated apart from one another to increase the distance between the jaws. Presuming the serrated lugs 60 are not engaged with one another, the finger holds 20, 20 may be repositioned in opposite directions to increase the distance between the jaws 12, 14. But where the serrated lugs 60 are engaged with one another to lock the relative positions of jaws 12, 14 in a compressive manner around the implant head 120, the serrated lugs 60 must first be vertically repositioned, such as by manipulating the finger holds 20, 22, so that the detent surfaces 64 of one serrated lug clear the detent surfaces of the other serrated lug. After the lugs 60 have been vertically repositioned to clear one another, the finger holds 20, 20 may be repositioned in opposite directions to increase the distance between the jaws 12, 14.

While the foregoing exemplary forceps 10 has been described with the first arm 16 including a dual prong jaw 12, whereas the second arm 18 includes a single prong jaw 14, it is also within the scope of the disclosure for the jaws 12, 14 to be interchangeable and removably coupled to the arms. In addition, it is within the scope of the disclosure for each arm 16, 18 to include a singe prong jaw 12. Likewise, it is within the scope of the disclosure for each arm 16, 18 to include a double prong jaw 14. In this manner, the forceps 10 may include two, three, or four prongs depending upon which jaws 12, 14 are mounted to the arms 16, 18.

It should also be noted that one or more of the jaws 12, 14 may be coated with an anti-slipping material in order to inhibit rotation of semispherical objects, for example, grasped by the forceps 10. Exemplary anti-slipping materials include, without limitation, nylon and rubber. Those skilled in the art are familiar with application of a nylon or rubber coating to a metallic object. Hence, a detailed explanation of coating the jaws 12, 14 with nylon or rubber has been omitted for purposes of furthering brevity.

Referencing FIG. 11, a second exemplary surgical instrument 210, generally referred to hereafter as a forceps, includes opposed jaws 212, 214 that are repositionable with respect to one another to accommodate and retain different sized objects. Each jaw 212, 214 is mounted to a proximal end of a respective arm 216, 218 that terminates in finger holds 220, 222 at a proximal end. The arms 216, 218 are pivotally coupled to one another via a pivot pin 224 in order to allow the relative positions of the jaws 212, 214 to be changed by manipulating the finger holds 220, 222. In this exemplary embodiment, the arms 216, 218 are pivotally coupled to one another via a box hinge 226. However, those skilled in the art will understand that hinge arrangements other than a box hinge may be used with the exemplary forceps 210 such as, without limitation, a scissor hinge.

Each arm 216, 218 includes a rectangular cross-section extending between the finger holds 220, 222 and box hinge 226, as well as between the box hinge and the jaws 212, 214. The cross-section of the arms 216, 218 changes along the length of each arm. By way of example, the cross-section of each arm 216, 218 at the proximal end, proximate the finger holds 220, 222, is dominated by larger top and bottom surfaces 228, 230 in comparison to smaller left and right side surfaces 232, 234. But the cross-section changes so that proximate the box hinge 226, the top and bottom surfaces 228, 230 are only slightly larger than the left and right side surfaces 232, 234. On a proximal end of the box hinge 226, the top and bottom surfaces 228, 230 have approximately the dimensions as the left and right side surfaces 232, 234, while these dimensions decrease proportionally until reaching the distal end proximate the jaws 212, 214.

The box hinge 226 comprises a female portion that includes corresponding rails 236, 238 of the first arm 216 that define a gap 240 therebetween. Each rail 236, 238 is integrally mounted to proximal and distal segments 242, 244 of the first arm 216, which are operative to longitudinally offset the proximal segment from the distal segment of the first arm. Both rails 236, 238 include an obtuse angled corner 246 that is laterally offset from the proximal and distal segments. Though not required, each rail 236, 238 includes substantially planar top and bottom surfaces 248, 250 that are separated from one another by a constant dimension, evidenced by a uniform material thickness between the top and bottom surfaces. The gap 240 between the rails 236, 238 is partially occupied by a male portion of the second arm 218 that includes a beam 252 formed by reducing the thickness of the second arm along a predetermined portion of the arm. This reduction in thickness in the second arm 218 may be accomplished by forging or grinding away material to form two generally rectangular cutouts 254. In this exemplary embodiment, the depth of the cutouts 254 is approximately the same as the thickness (i.e., distance between top and bottom surfaces 248, 250) of each rail 236, 238. Both the rails 236, 238 and the beam 252 include a through hole (not shown) that receives the pivot pin 224, thereby allowing the rails to pivot with respect to the beam and, thus, the arms 216, 218 to pivot with respect to one another. As discussed previously, the proximal and distal segments 242, 244 of the first arm 216 are longitudinally offset, whereas the second arm 218 is generally longitudinally aligned. As a result, when the arms 216, 218 are pivoted with respect to one another to close the jaws 212, 214, the distal ends of the arm 216, 218 are substantially parallel and adjacent.

Opposite the proximal ends, both arms 216, 218 include serrated lugs 260 that protrude toward one another in an inverted orientation. The serrated lugs 260 include a series of ramps 262 interposed by detent surfaces 264 that provide for a ratcheting action when the lugs engage one another. When the serrated lugs 260 engage each other, the lugs are operative to maintain the jaws 212, 214 in a predetermined position and inhibit the jaws from being opened. As will be discussed in more detail hereafter, the serrated lugs 260 are operative to lock the jaws 212, 214 in compression around various sized objects such as, without limitation, a semispherical implant.

Referring to FIGS. 2-4 and 11, both jaws 212, 214 are substantially the same as the jaw 14 of the first exemplary embodiment. Accordingly, a redundant description of the jaws 212, 214 has been omitted in furtherance of brevity.

The forceps 210 may be manufactured from any material. In exemplary form, the forceps 210 are manufactured from a metal in order to create a reusable tool. Exemplary metals include, without limitation, stainless steel, titanium, and nickel. Alternately, the forceps 210 may be manufactured using a plastic material for immediate disposal after one use. And the forceps may be also manufactured from a ceramic.

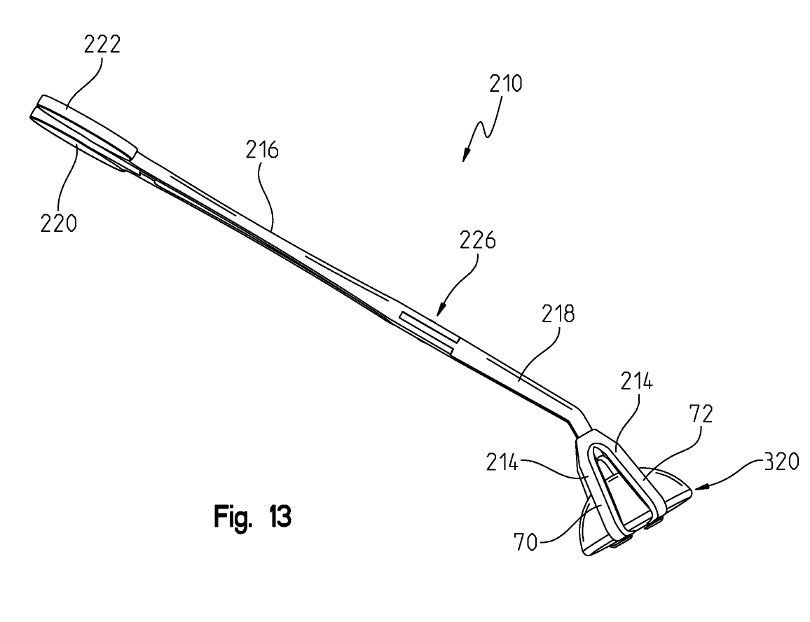

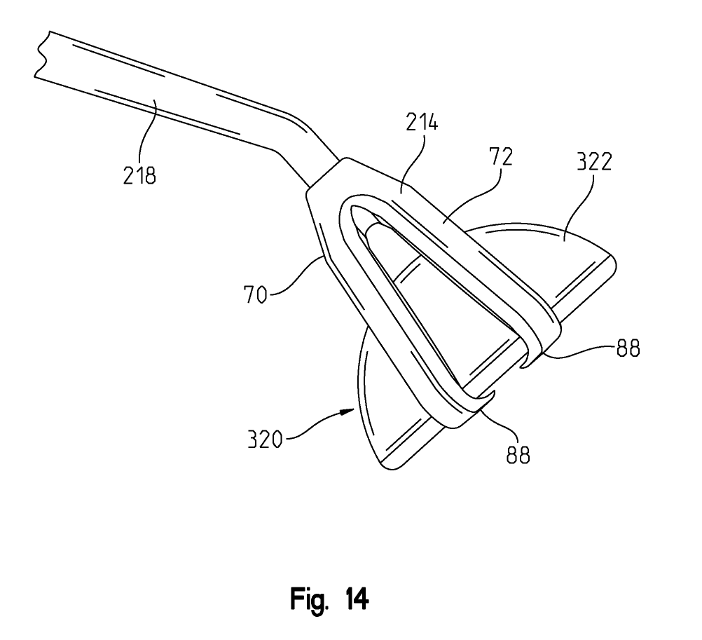

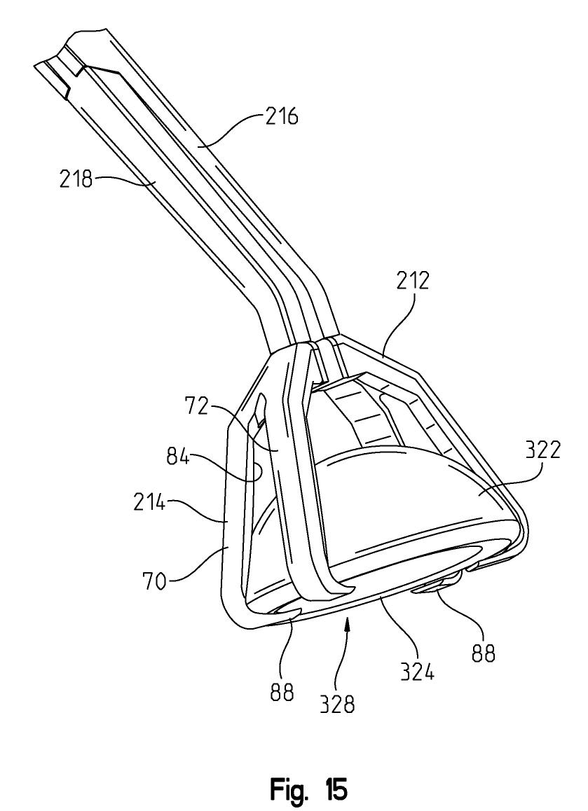

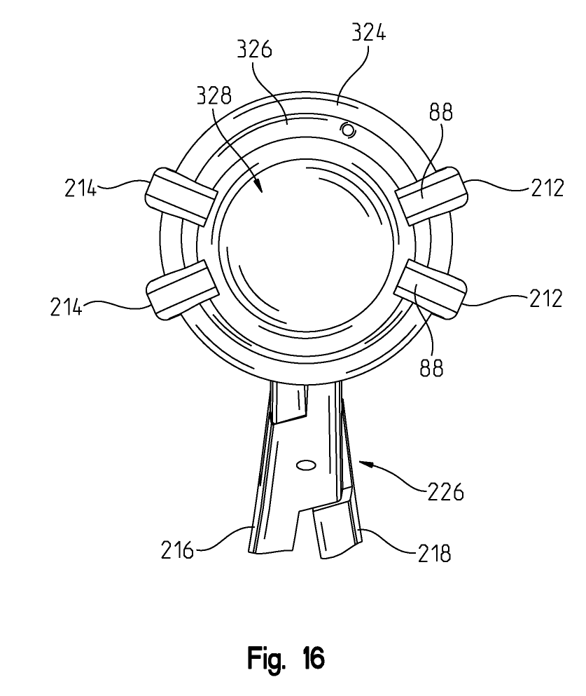

Referring to FIGS. 11-16, in use, the exemplary forceps 210 are opened so that the jaws 212, 214 are spaced apart from one another in order to grasp an object 320. In exemplary form, this object may comprise a semispherical or spherical implant head 320, such as those used for hip joint replacement and shoulder joint replacement surgeries. The finger holds 220, 222 may be accept a finger and thumb of a user (not shown) so that the finger holds 220, 222 are moved away from one another in order to correspondingly move the jaws 212, 214 away from one another. After the jaws 212, 214 are sufficiently spaced apart to accommodate the object in question, the jaws are positioned so that the object 320 interposes the jaws.

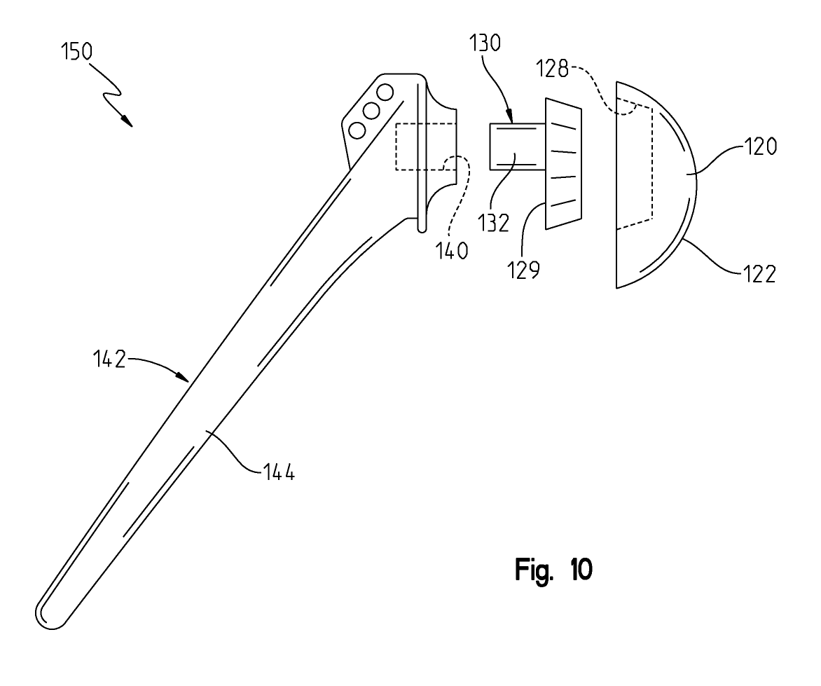

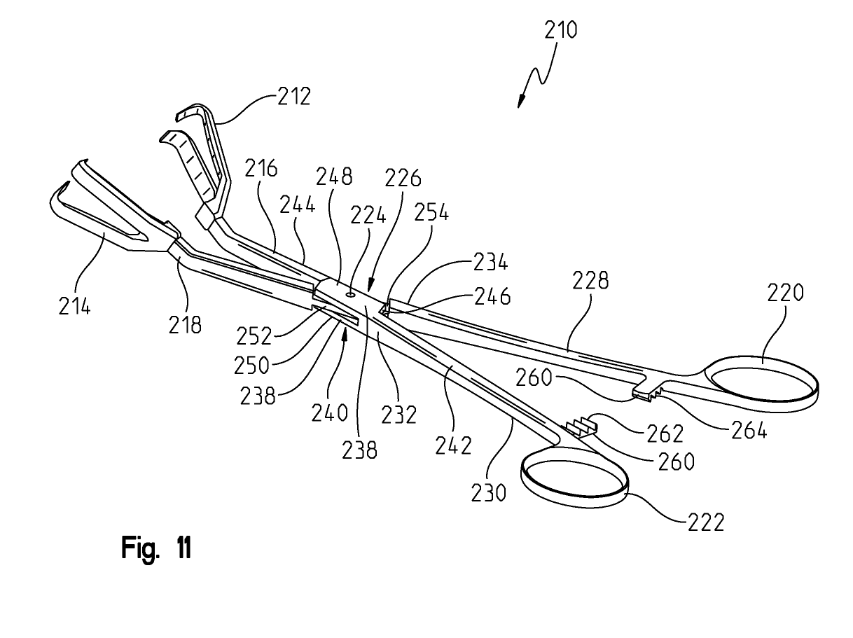

Specifically, in the context of a semispherical implant head 320, the spherical part 322 of the implant head is oriented to face the proximal end of the forceps 210 so that the spherical part contacts the third section 84 of each prong interior surface 78. In exemplary form, the underside of the implant head is relatively flat with a rounded-over circumferential lip 324 being outset from a recessed circumferential ring 326. This circumferential ring 326 generally defines an opening 328 that receives a taper adapter 130 (see FIG. 10) having a tapered projection extending therefrom.

After the implant head 320 is positioned between the jaws 212, 214, so that the spherical part 322 initially contacts the third section 84 of each prong interior surface 78, the jaws are moved toward one another to compress the implant head. When the spherical part 322 contacts the third sections 84 of the prongs 70, 72, a tapered wedge is created that forces the circumferential lip 324 toward the distal flanges 88 and eventually compresses the jaws 212, 214 circumferentially around the implant head 320. Specifically, the circumferential lip 324 is seated upon the distal flanges 88, while the spherical part initially contacts the third section 84 of each prong interior surface 78. In general, referencing the circumference of the implant head as a circle proximate the circumferential lip 324, the prongs are oriented and centered at approximately 35 degrees and 325 degrees for the first jaw 212, and at approximately 145 and 215 degrees for the second jaw 214.

Depending upon the size of the implant head 320, the jaws 212, 214 may retain the implant head in compression because the serrated lugs 260 have engaged one another. When the jaws 212, 214, and thus the finger holds 220, 222, are moved toward one another in close enough proximity, the sloped surfaces 262 of the serrated lugs 260 lugs ride against one another so that the detent surfaces 264 can engage one another and inhibit movement of the jaws 212, 214 (and finger holds 220, 222) further away from one another. At the same time, the angled surfaces 262 and detent surfaces 264 allow the jaws 212, 214 and the finger holds 220, 222 to be moved closer to one another (presuming no object between the jaws forecloses further movement) until reaching an endpoint. As a result, the serrated lugs 260 allow the jaws 212, 214 to be locked in a compression position, within a predetermined range of motion, while the implant head 320 is between the jaws 212, 214.

In order to release the implant head 320 from the jaws 212, 214, the finger holds 220, 222 are manipulated apart from one another to increase the distance between the jaws. Presuming the serrated lugs 260 are not engaged with one another, the finger holds 220, 220 are horizontally repositioned in opposite horizontal directions to increase the distance between the jaws 212, 214. But where the serrated lugs 260 are engaged with one another to lock the relative positions of jaws 212, 214 in a compressive manner around the implant head 320, the serrated lugs 260 must first be vertically repositioned, such as by manipulating the finger holds 220, 222, so that the detent surfaces 264 of one serrated lug clear the detent surfaces of the other serrated lug. After the lugs 260 have been vertically repositioned to clear one another, the finger holds 220, 220 may be repositioned in opposite horizontal directions to increase the distance between the jaws 212, 214.

While the foregoing exemplary forceps 210 has been described with both arms 216, 218 including dual prongs, it is also within the scope of the disclosure for the jaws 212, 214 to include more than two prongs. In this manner, the forceps 210 may include more than four prongs depending upon how many prongs each jaw 212, 214 includes.

It should also be noted that one or more of the jaws 212, 214 may be coated with an anti-slipping material in order to inhibit rotation of semispherical objects, for example, grasped by the forceps 210. Exemplary anti-slipping materials include, without limitation, nylon and rubber. Those skilled in the art are familiar with application of a nylon or rubber coating to a metallic object. Hence, a detailed explanation of coating the jaws 212, 214 with nylon or rubber has been omitted for purposes of furthering brevity.

Following from the above description and invention summaries, it should be apparent to those of ordinary skill in the art that, while the methods and apparatuses herein described constitute exemplary embodiments of the present invention, the invention contained herein is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. A surgical forceps comprising:
a first repositionable arm that includes a first prong extending distally therefrom, the first prong including a base mounted to the first repositionable arm where the first prong extends laterally outward from the base and distally away from the base, the first prong including a distal flange that extends laterally inward toward the base but stops short of overlapping the base, the first prong including a concave interior surface and a convex exterior surface, wherein the flange includes a widthwise dimension that is substantially greater than a thickness of the flange in a vertical direction;
a second repositionable arm that includes a second prong extending distally therefrom, the second prong including a base mounted to the second repositionable arm where the second prong extends laterally outward from the base and distally away from the base, the second prong including a distal flange that extends laterally inward toward the base but stops short of overlapping the base, the second prong including a concave interior surface and a convex exterior surface, wherein the flange includes a widthwise dimension that is substantially greater than a thickness of the flange in a vertical direction; and,
a third prong extending distally from the second repositionable arm, the third prong including a base mounted to the second repositionable arm where the third prong extends laterally outward from the base and distally away from the base, the third prong including a distal flange that extends laterally inward toward the base but stops short of overlapping the base, the third prong including a concave interior surface and a convex exterior surface, wherein the flange includes a widthwise dimension that is substantially greater than a thickness of the flange in a vertical direction;
wherein an end of the flange of the first prong is oriented generally in the direction of the end of the flange of the second and third prongs;
wherein the convex exterior surface of the first prong is oriented generally away from the convex exterior surface of the second and third prongs;
wherein the concave interior of the first prong is oriented generally toward the concave interior of the second and third prongs; and,
wherein an end of the flange of the third prong is oriented generally in the direction of the end of the flange of the second and third prongs.

2. The surgical forceps of claim 1, wherein at least one of the first repositionable arm and the second repositionable arm includes a finger loop mounted thereto in order to reposition the arms with respect to one another.

3. The surgical forceps of claim 1, wherein the first repositionable arm and the second repositionable arm each includes a finger loop mounted thereto in order to reposition the arms with respect to one another.

4. The surgical forceps of claim 1, wherein the first repositionable arm is pivotally repositionable with respect to the second repositionable arm about a common axis, and the first repositionable arm and the second repositionable arm cooperate to form a box hinge.

5. The surgical forceps of claim 1, wherein the second and third prongs are rotationally offset from one anther between ten and ninety degrees.

6. The surgical forceps of claim 1, wherein the first and second prongs are rotationally offset from one anther between forth-five and three hundred and twenty ninety degrees.

7. The surgical forceps of claim 1, further comprising a fourth prong extending distally from the first repositionable arm, the fourth prong including a base mounted to the first repositionable arm where the fourth prong extends laterally outward from the base and distally away from the base, the fourth prong including a distal flange that extends laterally inward toward the base but stops short of overlapping the base, the fourth prong including a concave interior surface and a convex exterior surface, wherein the flange includes a widthwise dimension that is substantially greater than a thickness of the flange in a vertical direction.

8. The surgical forceps of claim 7, wherein the first and fourth prongs are rotationally offset from one anther between ten and ninety degrees.

9. The surgical forceps of claim 7, wherein:
the concave interior surface of the first prong includes a planar segment acutely angled with respect to a longitudinal axis extending through the first arm; and,
the concave interior surface of the second prong includes a planar segment acutely angled with respect to a longitudinal axis extending through the second arm.

10. The surgical forceps of claim 1, wherein:
the concave interior surface of the second prong includes a planar segment acutely angled with respect to a longitudinal axis extending through the first arm; and,
the concave interior surface of the third prong includes a planar segment acutely angled with respect to a longitudinal axis extending through the second arm.

11. The surgical forceps of claim 1, wherein:
the concave interior surface of the first prong includes a planar segment acutely angled with respect to a longitudinal axis extending through the first arm; and,
the concave interior surface of the second prong includes a planar segment acutely angled with respect to a longitudinal axis extending through the second arm.

12. The surgical forceps of claim 1, wherein the concave interior surface of the first prong includes a first planar segment adjoining a second planar segment adjoining a convex interior surface.

13. The surgical forceps of claim 12, wherein:
the concave interior surface of the second prong includes a first planar segment adjoining a second planar segment adjoining a convex interior surface; and,
the convex interior surfaces of the first and second prongs face one another.

14. A forceps comprising:
a first arm and a second arm pivotally connected to each other, each of the first and second arms having opposing ends;
a first jaw coupled to the first arm, the first jaw including at least one laterally outward bowed prong at least partially defining an interior concavity, the at least one laterally outward bowed prong includes a distal flange extending medially in a first plane; and
a second jaw coupled to the second arm opposite the second finger hold, the second jaw including at least one medially outward bowed prong at least partially defining the internal concavity, the at least one medially outward bowed prong includes a distal flange extending laterally in the first plane.

15. The forceps of claim 14, further comprising a first finger hold and a second finger hold, each of the first and second finger holds being coupled to one of the opposing ends of the first and second arms, where the first finger hold and the second finger hold are repositionable relative to one another to pivotally reposition the first arm with respect to the second arm.

16. The forceps of claim 14, wherein the first arm and second arm are pivotally connected to each other using a box hinge.

17. The forceps of claim 14, wherein:
the at least one laterally outward bowed prong of the first jaw includes a concave interior surface and this concave interior surface includes a convex segment; and,
the at least one laterally outward bowed prong of the second jaw includes a concave interior surface and this concave interior surface includes a convex segment.

18. A forceps comprising:
a first arm mounted to and repositionable with respect to a second arm;
a first prong coupled to the first arm at a first junction having an X-axis that is perpendicular to a Y-axis that is perpendicular to a Z-axis that is itself perpendicular to the X-axis, the first prong extending proximally away from the first junction along the X-axis in a positive x-direction and laterally away from the first junction in the Y-axis in a positive y-direction, the first prong also including a distal flange that extends laterally toward the first junction in the Y-axis in a negative y-direction but does not overlap the first junction; and,
a second prong coupled to the second arm at a second junction having the same coordinate system as the first prong, the second prong extending proximally away from the second junction along the X-axis in the positive x-direction and laterally away from the junction in the Y-axis in the negative y-direction, the second prong also including a distal flange that extends laterally toward the second junction in the Y-axis in the positive y-direction but does not overlap the second junction;
wherein the distal flange of the first prong extends toward the distal flange of the second prong.

19. The forceps of claim 18, further comprising a third prong coupled to the first arm at a third junction having the same coordinate system as the first prong, the third prong extending proximally away from the third junction along the X-axis in the positive x-direction and laterally away from the junction in the Y-axis in the positive y-direction, the third prong also including a distal flange that extends laterally toward the third junction in the Y-axis in the negative y-direction but does not overlap the third junction.

20. The forceps of claim 19, further comprising a fourth prong coupled to the second arm at a fourth junction having the same coordinate system as the first prong, the fourth prong extending proximally away from the fourth junction along the X-axis in the positive x-direction and laterally away from the junction in the Y-axis in the negative y-direction, the fourth prong also including a distal flange that extends laterally toward the fourth junction in the Y-axis in the positive y-direction but does not overlap the fourth junction, wherein the distal flanges of the first and third prongs extend toward the distal flanges of the second and fourth prongs.

21. A surgical forceps comprising:
a first leg repositionable with respect to a second leg, the first leg having mounted thereto a first hook, the first hook including a convex exterior portion and a concave interior portion, the concave interior portion of the first hook partially defining an internal cavity, the second leg having mounted thereto a second hook, the second hook including a convex exterior portion and a concave interior portion, the concave interior portion of the second hook partially defining the internal cavity, wherein the concave interior portions of the first and second hooks face one another.

22. The surgical forceps of claim 21, wherein the concave interior portion of at least one of the first hook and the second hook includes a convex interior portion.

23. The surgical forceps of claim 21, wherein:
the first leg has mounted thereto a third hook, the third hook including a convex exterior portion and a concave interior portion, the concave interior portion of the third hook partially defining the internal cavity; and,
the first hook and third hook are radially offset from one another between ten and ninety degrees.

24. The surgical forceps of claim 23, wherein the concave interior portions of the first and third hooks face the convex interior portion of the second hook.

25. The surgical forceps of claim 23, wherein:
the second leg has mounted thereto a fourth hook, the fourth hook including a convex exterior portion and a concave interior portion, the concave interior portion of the fourth hook partially defining the internal cavity; and,
the second hook and fourth hook are radially offset from one another between ten and ninety degrees.

26. The surgical forceps of claim 25, wherein the first hook and the fourth hook are radially offset from one another between eighty and two hundred and eighty degrees.

27. The surgical forceps of claim 25, wherein the concave interior portions of the first and third hooks face the convex interior portions of the second and fourth hooks.
